(19) United States
(12) Patent Application Publication
(10) Pub. No.: US 2019/0216637 A1
(43) Pub. Date: Jul. 18, 2019

(54) CRYOTHERAPY MACHINE FOR RECOVERY OR TREATMENT OF THE MUSCULAR SYSTEM BY HOMOGENEOUS COLD APPLICATION

(71) Applicant: QW HEALTH COLOMBIA S.A.S., Bogota (CO)

(72) Inventor: Carlos Alberto Sierra Murillo, Bogota (CO)

(73) Assignee: QW HEALTH COLOMBIA S.A.S., Bogota (CO)

(21) Appl. No.: 16/330,863

(22) PCT Filed: May 30, 2017
(86) PCT No.: PCT/IB2017/053173

§ 371 Date: Mar. 6, 2019

(51) Int. Cl. A61F 7/00; A61G 10/02; G16H 40/67

(52) U.S. Cl. CPC A61F 7/0053; A61G 10/02; A61F 2007/0094; A61F 2007/0064; A61F 2007/0093; G16H 40/67

(57) ABSTRACT

The present invention relates to a device for carrying out cryotherapy treatments, which corresponds to a closed element, shaped like a tube or container, which mainly includes a treatment chamber into which the patient enters and is closed by an access door, as well as a system for supplying and storing a treatment gas, i.e. nitrogen, which allows to reach very low temperatures that are adequate for the recovery of the patient's muscular system. In this way, the device of the present invention has the advantage that the bottom thereof has a floor in the form of a grid, which allows that the gas used and applied to the patient to pass through same and then to be recovered by a feedback system that recirculates the gas and sends it back to the top of the device for application.

### CROSS REFERENCE TO RELATED APPLICATION

This application is a national stage entry of PCT/162017/053173 filed May 30, 2017, under the International Convention.

### TECHNICAL FIELD

The present invention relates to a device for carrying out cryotherapy treatments, which corresponds to a closed element, with a shape similar to a tube or container, which mainly has a treatment chamber where the patient is entered and is closed by means of an access door, as well as a treatment gas supply and storage system, i.e., nitrogen, which allows to reach very low temperatures, which are adequate to perform the recovery of the muscular system in the patient. Thus, the device of the present invention has the advantage that has in its lower part a floor in the form of a grid, which allows that the gas used and applied to the patient to pass through the grid and be recovered by a feedback system that recirculates the gas and sends it back to the top of the device for application. Additionally, the device of the present invention allows a more suitable treatment and more comfortable for the patient, since the device includes a cold gas supplying mechanism that is distributed throughout the entire treatment chamber, that is, the cold gas is injected through a series of openings arranged throughout the length or height of the chamber in order that the distribution of the gas is completely uniform and the patient can feel it throughout his entire body simultaneously.

In addition, the device of the present invention has a series of devices and modules that allow constant real time monitoring of the operation of the same, while allowing communication by SMS or email with a remote station or a remote user, in order to determine when the device has been used or when the device has an inconvenience. Likewise, the device has removable upholstery, which allows to wash it according to the use, ensuring hygiene and allowing the user to have new or clean upholstery every time the device is use.

### BACKGROUND OF THE INVENTION

Currently, in the sports field or in any field related to physical activity and performance, it is required the recovery of the person who practices it in a fast and adequate manner, especially when performing high-level competitions or professionally, since these athletes must have a specific performance and also their bodies must be adapted to continuously and constantly meet these activities.

In this way, the use of cryotherapy, cold treatment at very low temperatures for the recovery of the muscular system, is a widely used practice that provides very good results, since it allows the person to have an adequate muscular recovery in a very short time, undergoing continuous treatments of a few minutes, usually less than 10 minutes, which help the muscles to contract and recover, in order to carry out the sport activity again and have the expected performance.

Thus, in the state of the art, there is a plurality of disclosures related to cryotherapy devices, among them the document DE 3641293, which is related to a device for carrying out a whole-body cryotherapy procedure in a patient in need thereof for rheumatic disorders, wherein the device is composed of a cabin where the patient is accommodated, a source of cold gas in the form of liquid nitrogen, which evaporates in a container of liquid gas and is passed to the interior of the cabin, and a device for supplying the patient with breathing air, wherein the breathing air supplying device to the patient is arranged in an upper region of the cabin by a partition wall which isolates the patient by the region of the neck, where this partition has an adjustable height according to the requirements of the patient.

However, the device described above has the disadvantage that the patient is located inside but does not have any vertical gas feedback system and its floor is solid avoiding the gas to pass through, so the cold is concentrated in the lower part corresponding to the feet, so the temperature inside is not distributed homogeneously. Besides, this device can be considered as “conventional”, where the upholstery is fixed and does not ensure proper hygiene and cleaning for all users.

On the other hand, document U.S. Pat. No. 4,880,003 discloses a cabin to carry out a full body cryotherapy process with a low temperature gas, where the cabin has an open roof for the supply of cold gas, where the distance from the upper edge of the walls of the cabin and the floor can be adjusted to the height of the patient's neck. Likewise, the cabin has walls of an insulating material, a door for the entrance to the cabin, a series of emanation devices for the treatment gas mounted on the walls, a return arrangement device for the treatment gas (horizontal feedback). In one embodiment, the floor of the cabin is not designed as a grid by which an opening could be provided. However, this invention has the problem that the supply of gas for cooling is made from the top to the bottom, but not in a homogeneous way, but depends on the properties of the gas to autonomously flow down, which it does not ensure homogeneity and with its solid floor promotes a gas and temperature concentration around the feet. Likewise, the use of removable upholstery is not suggested, but merely insulating walls are mentioned, but they cannot be removed for cleaning.

However, the device described above has the disadvantage that the patient is located inside but does not have any vertical gas feedback system and its floor is solid avoiding the gas to pass through, so the cold is concentrated in the lower part corresponding to the feet, so the temperature inside is not distributed homogeneously. Besides, this device can be considered as “conventional”, where the upholstery is fixed and does not ensure proper hygiene and cleaning for all users.

On the other hand, document U.S. Pat. No. 4,880,003 discloses a cabin to carry out a full body cryotherapy process with a low temperature gas, where the cabin has an open roof for the supply of cold gas, where the distance from the upper edge of the walls of the cabin and the floor can be adjusted to the height of the patient's neck. Likewise, the cabin has walls of an insulating material, a door for the entrance to the cabin, a series of emanation devices for the treatment gas mounted on the walls, a return arrangement device for the treatment gas (horizontal feedback). In one embodiment, the floor of the cabin is not designed as a grid by which an opening could be provided. However, this invention has the problem that the supply of gas for cooling is made from the top to the bottom, but not in a homogeneous way, but depends on the properties of the gas to autonomously flow down, which it does not ensure homogeneity and with its solid floor promotes a gas and temperature concentration around the feet. Likewise, the use of removable upholstery is not suggested, but merely insulating walls are mentioned, but they cannot be removed for cleaning.

Also, DE 4316764 discloses a device for performing full-body cryotherapy in a patient, which incurs low operating costs, allows the treatment of the entire patient, and ensures high-temperature consistency during the treatment, wherein the device allows the supply of cold air to a therapy chamber by at least one cooling system which is operated electrically without liquefaction, without air storage, and without the addition of additional gases. The device also includes a cabin where the patient is admitted in which there is a device to circulate the cold air through a material of the walls and using the air heat exchanger.

However, as the same of the previously mentioned inventions, the device described in said patent has the disadvantage that includes a solid floor and without any type of vertical feedback system, while the gas supply is only made from the top, carrying to the problem related to the accumulation of gas in the lower part of the chamber where the patient is located, which can be very uncomfortable for the user and can cause frostbite.

In addition, US 2015094702 which refers to a cryogenic device for carrying out a whole body cryotherapy of a patient, where the device includes a treatment cabin to receive a patient, the treatment cabin has closed walls, a closed roof, and a door for entry to the treatment cabin; a cold treatment gas preparation and distribution system to prepare a cold gas mixture and introduce it into the treatment cabin; and a control system for controlling the introduction of the treatment gas from the distribution and preparation system to the treatment cabin, wherein the treatment cabin further includes a breathing window to allow the patient located in the interior to breathe air from the exterior of the cabin, wherein said window includes sealing devices to allow airtight contact between the window and the patient's face. The cabin also includes a raised floor to adjust the distance between the floor and the patient's breathing window.

However, as with the previously mentioned documents, this invention presents a cryotherapy device such as those known in the market, which is also closed and only has a breathing window so that the patient can take air from the outside but that does not include an upper part, which is totally undesirable, and can create inconveniences in people suffering from some disorder or anxiety to close spaces (claustrophobia), has a solid floor, and the gas feeding is made by the upper part without vertical feedback, which does not ensure a homogeneous distribution of the low temperature throughout the patient's body.

Finally, US 2015265460 which teaches a system for carrying out cryotherapy, which comprises a closed cryogenic chamber with a door including heat insulation and a heat sensor, a refrigeration device with a condenser and a control and power unit, and a refrigerant supply line, wherein the refrigeration device has two cascades, and an evaporator with a fan installed in the ceiling or wall of the cryogenics chamber and connected by means of the supply line of the refrigerant to the double cascade refrigeration device, and where a heat control valve is installed at the inlet to the evaporator. The cryogenic chamber is equipped with a valve to equalize the pressure with the external environment. The system has a control console with a sensor panel installed on the external surface of the cryogenics chamber.

However, this invention has a common cryotherapy device as known in the art, where the device has a fan and a series of sensors to control the heat entering the device and thus be able to keep the cold, but did not suggest or mention a homogeneously gas supply system, does not have a retro feeding system or means to control or monitor the device remotely.

In accordance with the above, and taking into account the devices for cryotherapy or muscle recovery system existing in the state of the art, there is a problem related to the supply of the cooling gas in the cooling chambers of the cryotherapy systems or devices, since in all cases there is a supply made from the top to the bottom, where this supply is carried out thanks to the nature of the cooling gas, while the feedback of the supplied gas is not carried out or its just horizontal feedback but never a vertical feedback; thus, there are unnecessary and high operation expenses. Additionally, it is important to take into account the hygiene of existing devices, since they do not have any type of removable element that allows cleaning after each use, which is totally undesirable and can lead to health problems in some patients. In addition to the above, the problem arises that it is not possible to perform constant and real-time monitoring of existing devices in the state of the art, which allows these devices to be operated in an arbitrary and uncontrolled manner, thus leading to costs of additional operations that cannot be monitored, while on-site expert assistance is required when carrying out maintenance or repair operations if required.

In this way, a person skilled in the art can clearly see that there is a need to design and implement a cryotherapy system or device, which allows overcoming the previously mentioned problems, thus, it has some type of system that allows to reuse the cooling gas supplied, improve the temperature distribution avoiding gas concentration around the feet and reduce operating costs, while it can be adequately cleaned assuring its hygiene, and more importantly, that it has a module or mechanism that allows constant monitoring, and allows a station to be informed or remote user by different means when the device has been used and even when it comes to manipulating or altering the device.

### BRIEF DESCRIPTION OF THE FIGURES

FIG. 1 shows a top perspective view of the cryotherapy device of the present invention.

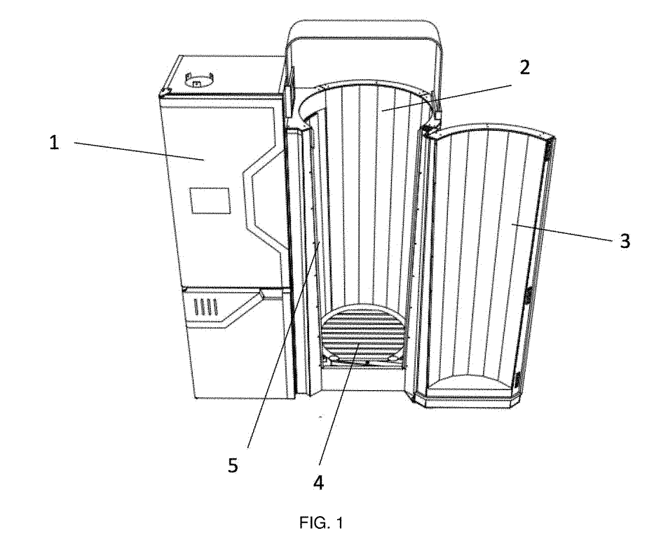

FIG. 2 shows a top view of the cryotherapy device of FIG. 1.

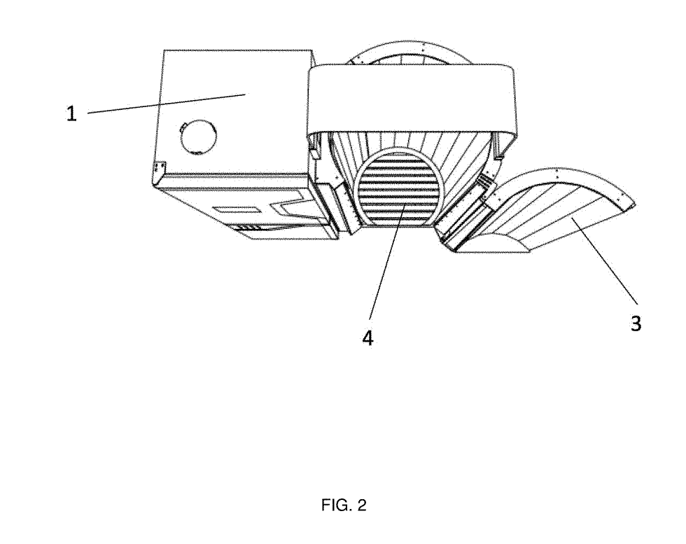

FIG. 3 shows a top perspective view of the device of the present invention showing in a detailed manner how it detaches the upholstery attached to the door and cooling cabin.

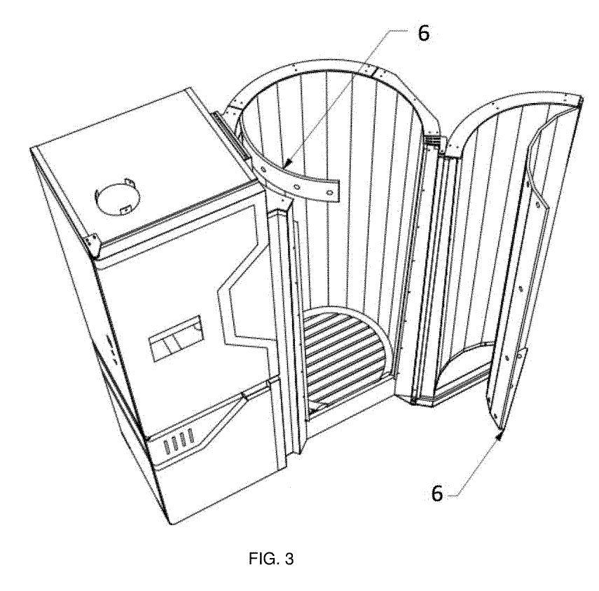

FIG. 4 shows a side view of the device of the present invention, showing the cooling gas supply diffuser arranged over the entire height of the cooling cabin.

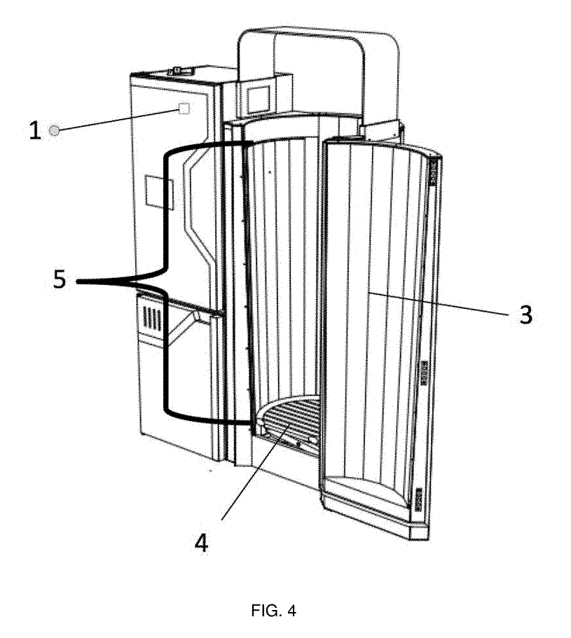

FIG. 5 shows a front view of the device of the present invention with the floor lift system of the cooling cabin in the raised position, thus showing the scissor-type mechanism that allows said elevation to be made.

FIG. 6 shows a block diagram of the operation of the device of the present invention and the communication and notification system for monitoring said device.

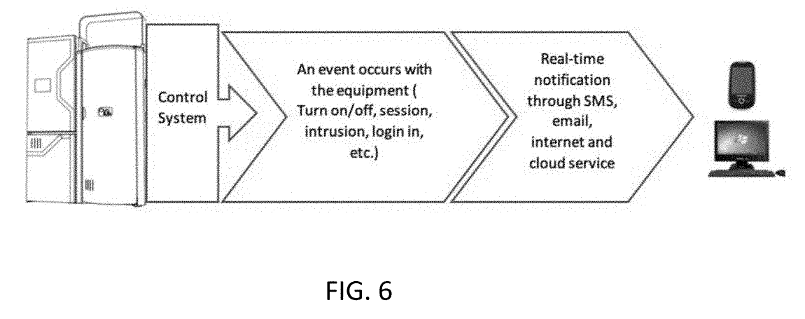

### DETAILED DESCRIPTION OF THE INVENTION

The present invention relates to a device for carrying out cryotherapy treatments in patients in need of recovery of the muscular system, which comprises the following components or parts:

A control system (1) located on one of the sides of the device that includes all the elements that allow the gas supply, the pumping, the control, and other elements that ensure the operation and that correspond to widely known elements in the state of the art and which are included in all existing cryotherapy devices;

A cooling cabin (2) having a cylindrical shape and which the user is located inside it to receive the treatment;

An access door (3) located in the front of the cabin (2) which closes said cabin (2) and allows or limits the user's access to perform the treatment;

A floor or bottom (4) located, as its name indicates, in the lower part of the cooling cabin (2), on which the user who is going to receive the treatment is placed, where said floor or bottom (4) has a plurality of slots or holes that can convert it into a grid or similar element;

A gas supply system (5) or diffuser, which is arranged inside the cooling cabin (2) uniformly along the entire height of said cabin (2), wherein said delivery system or diffuser (5) corresponds to a plurality of holes spaced and located in the internal walls of the cabin (2) that allow a homogeneous diffusion or supply of the cooling gas;

A vertical feedback system (not shown) located inside the control system (1), which is directly connected to the floor (4) for the reception of the gas, and which allows sending the received gas back to the supply system or diffuser (5), allowing maximum use of the cooling gas and preventing the undesirable gas accumulation around the user feet;

An upholstery (6) covering the inner side walls of the cooling cabin (2) and the internal part of the door (3), where said upholstery is removable and has a plurality of attach points on its side and upper part which adhere to the walls of the cabin (2), in order to allow said upholstery (6) to be easily and adequately removed;

A lifting mechanism (7) located below the floor or bottom (4) and that allows raising said floor or bottom (4) to adjust the height of the cooling cabin (2) to the height of each patient receiving the therapy, wherein said lifting mechanism (7) corresponds to a scissor-type mechanism; and

A communication system (not shown) located inside the control system (1), which has a wireless communication module that allows notifications to be sent to a remote user when there is something new with the cryotherapy device, or when the device has been tampered in an unauthorized manner. The communication system can be turn on or off remotely.

Thus, the cryotherapy device of the present invention presents a plurality of advantages with respect to the commercially available devices existing in the state of the art, mainly with regard to issues of improving the gas and temperature homogeneous distribution, saving operating and maintenance costs, and with regard to supply of proper hygiene for the operation in the field of muscular system recovery or management of patients of different types.

Thus, first, the floor or bottom (4) of the present invention is perforated, that is, it has a plurality of holes disposed over the entire surface thereof, which allows constant communication between the interior of the cooling cabin (2) and the bottom of the floor or bottom (4) so that the cooling gas can pass through said floor or bottom (5). Thus, the holes disposed on the floor or bottom (4) can correspond to the grooves that allow obtaining a floor (4) in the form of a grid, which allows the user to experience a more homogeneous cold, since the gas does not accumulate in the lower part, as is presented in conventional devices existing in the art, while allowing a more pleasant and less uncomfortable feeling of the cold. In this way, the cold differential between the calves and the abdomen of the user that is located inside the cooling cabin (2) is minimal, which allows to obtain a much greater tolerability of the cold, and therefore, greater comfort for the user.

In a preferred embodiment, the device has a gas collection mechanism located exactly below the floor or bottom (4), which allows to collect the gas that passes through the plurality of holes or grooves in the floor or bottom (4) and re-feed it by means of the feedback system, in order to enter it again in the cabin (2) by using the diffuser (5).

Now, the delivery system or diffuser (5) corresponds to a mechanical barrier located inside the cabin (2), preferably on one of the sides as shown in the figures, where said diffuser (5) is arranged from the upper part of the cabin (2) to the floor or bottom (4), thus allowing the flow of gas to diffuse and tangentially exit to the person inside the cabin (2), allowing that the cooling gas is distributed more evenly and homogeneously.

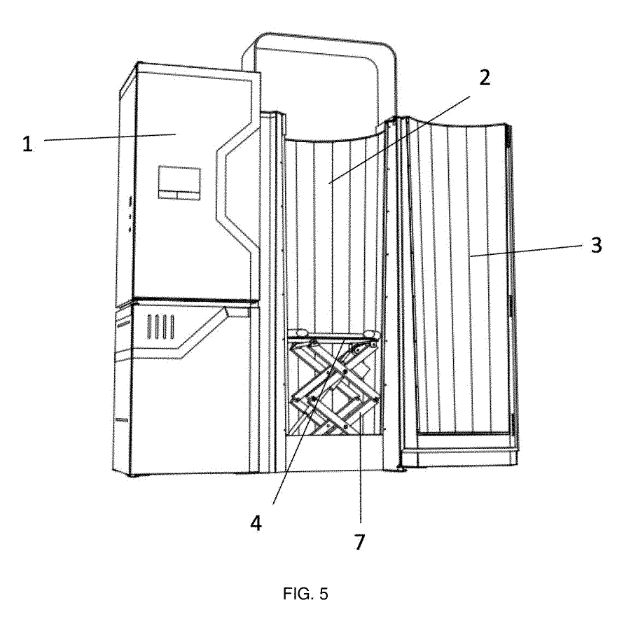

In a preferred embodiment, the device of the present invention has a lifting system or mechanism (7) located in the lower part of the floor or bottom (4) that corresponds to several crossed bars as illustrated in detail in FIG. 5. Said crossbars form a scissor-type mechanism, which allows to raise the floor or bottom (4) in a more robust and suitable way to support any type of user that is located inside the cooling cabin (2) without presenting problems with the operation of the device in general and adjusting the height to the user.

On the other hand, the device has a system of notifications, which allows the notification via email or SMS (text message) to at least one remote user, where notifications can be configured according to the requirements that they are needed, from a warning of when the device is turned on or off, to notifications of intrusion or tampering of the same, including announcements of start or end of session, among others. Thus, there is a control center inside the control system (1) that monitors the device constantly and allows you to see reports directly on the device or can be sent to authorized users through the wireless communication module already inside the notification system and the reports can be see over the internet.

In another embodiment of the invention, the cryotherapy device has a session configuration mechanism, which allows, by an input interface device, to enter the parameters to control the intensity and duration of each session, where it is possible to vary both the temperature inside the cooling cabin (2) and the time in which the therapy session is carried out. All the above is done by a controller that receives the information entered and controls the mechanical elements inside the control system (1) to know the amount of gas to be supplied by means of the supply system or diffuser (5) and the time that these elements are going to be on during a specific session.

1. A cryotherapy device comprising:
a control system (1) located on a side of the device;
a cooling cabin (2);
an access door (3) located in a front end of the cabin (2);
a perforated floor or bottom (4) located in a lower part of the cooling cabin (2), the perforated floor or bottom (4) including a plurality of grooves or holes;
a gas supply system or diffuser (5) arranged inside the cooling cabin (2), the gas supply system or diffuser (5) is uniformly arranged along the entire height of said cabin (2);
a vertical feedback system located inside the control system (1), the feedback system having a first end connected to the perforated floor or bottom (4) and a second end connected to the gas supply system-or diffuser (5);
an upholstery (6) covering internal side walls of the cooling cabin (2) and an internal wall of the door (3);
a lifting mechanism (7) located below the perforated floor or bottom (4); and
a communication system located inside the control system (1), communication system includes a wireless communication module.

2. The cryotherapy device according to claim 1, wherein the cooling cabin (2) has a cylindrical shape.

3. The cryotherapy device according to claim 1, wherein the floor or bottom (4) has the form of a grid or grid.

4. The cryotherapy device according to claim 1, further including a gas collection mechanism located below the perforated floor or bottom (4).

5. The cryotherapy device according to claim 1, wherein the delivery system or diffuser (5) is a mechanical barrier with holes located inside the cabin (2), the mechanical barrier is arranged from the top of the cabin (2) to the perforated floor or bottom (4).

6. The cryotherapy device according to claim 1, wherein the removable upholstery (6) has a plurality of attach points on an upper part and sides, wherein the attach points adhere to the internal walls of the cabin (2) and the internal wall of the door (3).

7. The cryotherapy device according to claim 1, wherein the lifting system or mechanism (7) includes crossed bars forming a scissor-type mechanism.

8. The cryotherapy device according to claim 1, wherein the communication system performs communication by electronic mails or text messages (SMS).

9. The cryotherapy device according to claim 1, further including a session configuration mechanism to control intensity and duration time parameters of each session.
